(19) United States
(12) Patent Application Publication
(10) Pub. No.: US 2011/0288037 A1
(43) Pub. Date: Nov. 24, 2011

(54) Pharmaceutical Composition for the Treatment of Pathologies Caused by the General Response of the Immune System

(76) Inventors: Francesco Della Valle, Padova (IT); Maria Federica Della Valle, Padova (IT); Gabriele Marcolongo, Due Carrare (Padova) (IT); Gianpiero Ravagnan, Roma (IT); Vincenzo Di Marzo, Pozzuoli (Napoli) (IT)

(21) Appl. No.: 13/196,964

(22) Filed: Aug. 3, 2011

Related U.S. Application Data
(63) Continuation of application No. 11/724,501, filed on Mar. 14, 2007, now abandoned.

(30) Foreign Application Priority Data
Mar. 28, 2006 (EP) 06425206.7

(51) Int. Cl. A61K 31/16; A61P 37/00; A61P 37/08; A61P 17/00; A61P 27/02; A61P 25/00; A61P 17/06; A61P 29/00; A61P 19/04; A61P 13/08; A61P 9/10; A61P 25/02; A61P 15/00; A61P 31/12; A61P 1/00; A61P 1/04; A61K 31/7034

(52) U.S. Cl. 514/23; 514/625

(57) ABSTRACT

The present invention relates to a pharmaceutical composition consisting of amides of mono- and di-carboxylic acids and hydroxystilbenes, and may be used for the treatment of pathologies caused, sustained and/or characterised by an abnormal general response of the immune system, in both humans and animals.

### FIELD OF THE INVENTION

The present invention relates to a pharmaceutical composition comprising amides of mono- and di-carboxylic acids and hydroxystilbenes for the treatment of pathologies caused, sustained and/or characterised by an abnormal general response of the immune system, in both humans and animals.

### BACKGROUND ART

The literature is full of evidence of direct “cross-talk” between the cellular population consisting of mastocytes (M) and the general population of activated T lymphocytes (ATL).

Early evidence for the interaction between M and ATL cells began to emerge at the end of the 90s, from an Israeli immunology research group. In one interesting publication, the authors conclude by hypothesising that the “T cell-mast cell” interaction may be bidirectional and represent an important regulatory and/or modulatory stage of the general immune response (Mekori Y A et al. J. Allergy Clin. Immunol. 1999 September; 104:517-23). A direct interaction between the two cell lineages was shown for the first time in 2002; mastocyte mediators originating from the degranulation of human M cells are capable of increasing interferon-γ production by CD8+ and CD4+ T-lymphocytes (Francina L. de Pater-Huijsen et al.—Immunology 2002 May; 106:11-19). This fascinating hypothesis, reiterated in a “review” published in Nature Immunology in February 2005 (Galli S. et al.—Nature Immunology 2005 February; 6(2):135-42), is confirmed in the literature, in April 2005 when the same group, belonging to Stephen Galli, show the importance of degranulated TNF from M cells on T lymphocyte activation (Nakae S. et al. —Proc. Natl. Acad. Sci. USA 2005 April; 102(18):6467-72); the authors conclude by claiming the identification of a multiple mechanism by means of which mastocytes are capable of influencing the proliferation and production of cytokines from T lymphocytes. The direct “cross-talk” between M cells and T lymphocytes is only clearly confirmed and discussed in October 2005 (Salamon P. et al.—Allergy 2005 October; 60(10):1316-9) and its role in T lymphocyte mediated inflammatory processes demonstrated.

Currently, the diseases characterised by an abnormal immune response are treated by the administration of appropriate drugs which modulate lymphocyte activity exclusively.

The possibility of modulating the general immune response by modulating mastocytes and activated T lymphocytes simultaneously has never been proposed and described.

### SUMMARY OF THE INVENTION

Thus, the underlying idea behind the present invention is that of modulating the body's general immune response by simultaneously modulating mastocyte (M) reactivity—understood as being the differential degranulatory response of the same—and the activated T lymphocyte (ATL) cytokine and functional response.

### DETAILED DESCRIPTION OF THE INVENTION

Hence, the present invention relates to a pharmaceutical composition comprising amides of mono- and di-carboxylic acids and polyphenols belonging to the hydroxystilbene family, and may be used for the treatment of immuno-inflammatory diseases caused, sustained and/or characterised by abnormal general immune responses, in both humans and animals.

In particular, such diseases are those caused by exogenous antigenic stimulii (the so-called “non-self” stimulii). This includes, allergic rhinitis, bronchial asthma, allergic alveolitis, urticaria, atopic dermatitis, contact dermatitis, conjunctivitis and anaphylaxis. Around 10% of the global population suffer from allergic disorders, with an overall predominance in the industrialised countries, with spikes of 15% in sub-populations (infant atopic dermatitis).

Other types of disorders include those caused by endogenous antigenic stimulii (the so-called “self” stimulii), normally defined as autoimmune disorders, characterised by hypersensitivity towards body components. This includes multiple sclerosis, psoriasis, bullous pemphigus, urticaria pigmentosa, systemic scleroderma, uveitis, cicatricial ocular pemphigus, rheumatoid arthritis, systemic lupus erythematosus and psoriatic arthritis. The most frequently observed autoimmune diseases affect no less than 5% of the global population.

The disorders characterised and/or sustained by neuro-immunogenic inflammation—in practice almost all the inflammatory diseases—are organ-specific disorders such as interstitial cystitis in humans and in cats, prostatitis, arthrosis in humans and in dogs, numerous autonomic and somatic neuropathies, vulvovaginitis, vulvar vestibolitis, viral infections of the vagina and uterine neck, oral mucositis, Crohn's disease, ulcerative colitis, geriatric dermatitis (characterised by characteristic symptomatology such as dryness, dyskeratosis, itching, epidermal lesions etc), radiation dermatitis (from the sun, from radiotherapy in cancer patients etc.) and many others. Epidemiological estimates consider the incidence of such diseases to be no less than 40% of the population, and increasing rapidly due to increased average global lifespan and, in the field of veterinary medicine, in consideration of the increasing attention being paid to pets.

The mono- and di-carboxylic acid amides used in the pharmaceutical composition of the present invention are the N-acylderivatives of formula (I):

X—R₁—C(O)—Y₁ (I)

wherein X—R₁—C(O) is selected from the group consisting of:
a) a monocarboxylic acid acyl residue, wherein X—R₁ is a saturated or unsaturated alkyl radical having from 1 to 23 carbon atoms, and with 1 to 6 double bonds, or araliphatic, or aromatic, or heterocyclic, or heteroaromatic residue. The saturated or unsaturated alkyl radical may optionally be substituted with one or more hydroxy, amino, carbonyl, carboxyl, cycloalkyl, aryl, heterocyclic or heteroaromatic groups or condensed polycyclic groups. Examples of saturated or unsaturated aliphatic monocarboxylic acids, optionally substituted in the aliphatic chain with one or more of the above-mentioned groups include: acetic, propionic, butyric, caprylic, valeric, valproic, palmitic, oleic, stearic, lauric, myristic, arachidonic, deoxycholic acid and derivatives thereof having hydroxyl or amino groups as substituents of the aliphatic chain. Other examples include: glycolic, pyruvic, lactic, retinoic, hydroxyphenylacetic and alpha-lipoic acid. Preferred among the aromatic, heterocyclic or heteroaromatic monocarboxylic acids are: salicylic, acetylsalicylic, sulphosalicylic, benzoic, trimethoxybenzoic, nicotinic, isonicotinic, tenoic and fenylanthranylic acid;
b) the acyl residue of an aromatic, or araliphatic, or heterocyclic, or heteroaromatic, or saturated or unsaturated aliphatic dicarboxylic acid, with from 2 to 20 carbon atoms, optionally substituted with a hydroxy, amino, aromatic, heterocyclic or heteroaromatic group, wherein X is a residue of formula C(O)Y₂ and Y₂ may be —OH or have the same meaning as described for Y₁. When Y₂ is —OH the resulting carboxyl group may be salified with a biologically acceptable organic or inorganic cation, preferably sodium, potassium, magnesium, calcium or zinc.
   Examples of dicarboxylic acids include: fumaric, azelaic, trans-traumatic, succinic, glutaric, muconic, cromoglycic, malic, tartaric, aspartic, glutamic or phthalic acid.
Y₁ is selected from the group consisting of:
a) NH₂;
b) NR₂R₃, an amine residue of a mono or bifunctional aminoalcohol, wherein R₂ is an alcohol residue selected from a linear or branched C₁-C₂₀ mono or dihydroxyalkyl, optionally substituted in the alkyl chain with one or more aromatic groups, or a hydroxyaryl, optionally substituted with one or more linear or branched alkyl radicals with from 1 to 20 carbon atoms; or an amine residue of an aminoalkylaryl substituted in the aromatic ring with a methoxy group and an —OH group; the hydroxyl groups of such aminoalcohols may optionally be substituted with a phosphate or glycerophosphate group to form —OPO₃H₂ or —OPO₂H—O—CH₂—CH(OH)—CH₂—OH, or a glycoxy radical to form an O-glycoside, and preferred among the possible glycoxy radicals are, D- and L-glucose, D- and L-galactose, D- and L-mannose, D- and L-ribose, D- and L-fructose, D- and L-glucosamine, D-galactosamine, D-mannosamine, D-glucuronic acid and sialic acid; said O-glycosides may be α- or β-glycosides; R₃ is H, —CH₃ or the same as R₂;
c) an amino residue of an optically active or inactive aminoacid, wherein R2, together with the nitrogen atom to which it is bound, forms an alpha-aminoacid, optionally carrying a side chain optionally substituted with —OH, —OPO₃H₂, —OPO₂H—O—CH₂—CH(OH)—CH₂—OH, —SH, S—CH₃; R3 is —H or —CH₃
d) the amino residue of a glycosamine of formula (II):

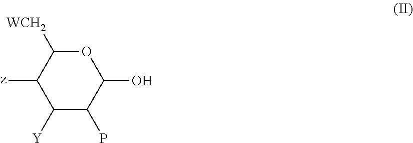

wherein one of the groups P, Y and Z is —N(H)— or —N(CH₃)— and the remaining groups are —H or —OH.

The mono- or dicarboxylic acid amides preferably used in the invention are N-acylderivatives of ammonia, ethanolamine, diethanolamine, 2-phosphoethylamine, 2-O-(beta-D-glucopyranosyl)aminoethanol, L-serine, glycine, 2-amino-1,3-propandiol, D-glucosamine, 4-hydroxy-3-methoxybenzylamine, N-methyl-2-aminoethanol, 2-hydroxypropylamine. The N-acylderivatives of 2-hydroxypropylamine may be optical isomers or a racemic mixture.

N-palmitoylethanolamine (N-PEA), N,N-bis(2-hydroxyethyl)-palmitamide, N-(2-hydroxypropyl)-palmitamide, N-(2-hydroxyethyl)-stearoylamide, N-(2-hydroxyethyl)-lauroylamide, N,N-bis(2-hydroxyethyl)-lauroylamide, N-(2-hydroxyethyl)-4-hydroxybutyramide, N-2-hydroxyethyl)-benzoylamide, N-acetylethanolamine, N-palmitoyl-D-glucosamine, N-palmitoyl-L-serine, 2-O-(beta-D-glucopyranosyl)-N-palmitoylaminoethanol, stearoylamide, oleoylamide, N-palmitoyl-2-amino-1,3-propandiol, N-palmitoyl-4-hydroxy-3-methoxybenzylamine, N,N′-bis(2-hydroxyethyl)nonandiamide, N,N¹-bis(2-hydroyethyl)-2-dodecendiamide, N,N′-bis(2-hydroxyethyl)fumaroyldiamide are advantageously used in the invention.

The hydroxystilbenes comprised in the pharmaceutical composition of the invention have the following structural formula (III):

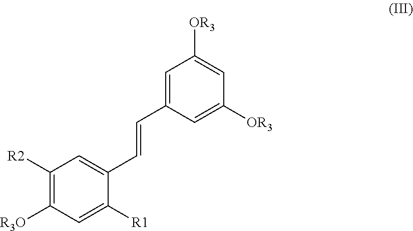

wherein:

R₁ is H, OH or R₂, and

R₂ is independently selected from H, OH, or linear or branched O—(C₁-C₆)alkyl;

R₃ is independently selected from H, linear or branched (C₁-C₆)alkyl, and a saccharide group selected from D- and L-ribose, D- and L-glucose, D- and L-galactose, D- and L-mannose, D-fructose, D- and L-glucosamine, D-galactosamine, D-mannosamine, glucuronic acid, N-acetyl-D-glucosamine, N-acetyl-D-galactosamine, N-acetyl-D-mannosamine.

When the substituent is (C₁-C₆)alkyl, it is preferably methyl, ethyl, n-propyl or iso-propyl.

When the substituent is a saccharide group forming a glycoside, this may be an α- or β-glycoside.

Preferably, R3 is independently hydrogen, or —CH₃, or is selected from: D- and L-ribose, D- and L-glucose, D- and L-galactose, D- and L-mannose.

Particularly preferred compounds of formula (III) are resveratrol and the glycosides of resveratrol.

The compounds of formula (III) may be in both the trans and cis isomeric forms.

The synthesis of said compounds of formulas (I) and (III) is well known in the art and is, for example, exemplified in EP 0 550 006, in US Patent Application 2004/0014682A1, in U.S. Pat. No. 5,506,224, in U.S. Pat. No. 5,618,842, in EP 0 751 947 and in EP 1 115 392

The one or more mono- or dicarboxylic acid amides and the one or more hydroxystilbenes are comprised within the composition, respectively, in quantities ranging from 0.0001 mg/kg/day to 20 mg/kg/day, preferably from 0.05 mg/kg/day to 10 mg/kg/day for the N-acylderivatives of aminoalcohols, and preferably from 0.005 mg/kg/day to 10 mg/kg/day for the hydroxystilbenes.

Said active ingredients, optionally micronised or co-micronised with one or more appropriately mixed parenteral, rectal, transdermal or topical administration, or in a form adapted for administration by inhalation or insufflation (either through the mouth or nose).

Said appropriately mixed active ingredients may be formulated for oral, buccal, parenteral, rectal, transdermal, topical administration onto the skin and mucosa, or in a form adapted for administration by inhalation or insufflation (either through the mouth or nose).

The pharmaceutical compositions for oral administration may be, for example, in the form of tablets or capsules (containing powders or oil mixtures), prepared in the conventional manner with pharmaceutically acceptable excipients such as binding agents (for example pre-gelatinised corn starch, polyvinylpyrrolidone or carboxymethyl-cellulose); fillers (for example lactose, microcrystalline cellulose or calcium hydrogen phosphate); lubricants (for example magnesium stearate, talc or silica); disintegrants (for example potato starch or sodium starch glycolate); or surfactants (for example sodium lauryl sulphate). Tablets may be coated, using methods well known in the art. Liquid preparations for oral administration may be, for example, in the form of solutions, syrups or suspensions or may be as lyophilised products to be reconstituted, prior to use, with water or other suitable carriers. Such liquid preparations may be obtained using conventional methods with pharmaceutically acceptable additives such as suspension agents (for example sorbitol syrup, cellulose derivatives or hydrogenated edible fats); emulsifiers (for example lecithin or acacia); non-aqueous carriers (for example almond oil, oily esters, ethyl alcohol or fractionated vegetable oils); and preservatives (for example methyl- or propyl-p-hydroxybenzoates or, sorbic acid).

Preparations may also appropriately contain flavourings, colourants, sweeteners and preservatives.

Preparations for oral administration may be appropriately formulated to allow the controlled release of the active ingredient.

Compositions for buccal administration may be in the form of conventionally formulated tablets or lozenges.

The appropriately mixed active ingredients may be formulated for parenteral administration by injection.

Formulations for injections may be presented in single dose form, for example in ampoules with added preservative. The compositions may be presented in the aforementioned form as suspensions, solutions or emulsions in oily or aqueous carriers and may contain formulary agents such as suspension agents, stabilisers and/or dispersants. Alternatively, the active ingredient may be in powder form for reconstitution, prior to use, using an appropriate carrier, for example sterile water.

According to the present invention, the active ingredients may also be formulated in rectal compositions such as suppositories or retention enemas, for example containing common basic suppository components such as cocoa butter or other glycerides.

In addition to the previously described compositions, the compounds may also be formulated as deposit preparations. Such long acting preparations may be administered as implants (for example subcutaneously, transcutaneously or intramuscularly) or by intramuscular injection. Thus, for example, the active ingredients may be formulated with suitable polymeric or hydrophobic materials (for example in the form of an emulsion in a suitable oil) or ion exchange resin or as sparingly soluble derivatives, for example as a sparingly soluble salt.

The appropriately formulated pharmaceutical compositions of the invention may be administered at dosages varying between 0.1 and 30 mg/kg, from 1 to 4 times per day. The dosage of the compositions will be determined according to the disease to be treated, the level of seriousness of said disease, and the condition of the patient. Furthermore, the dose will depend on the selected route of administration. It should be considered that it might be necessary to make continual adjustments to the dosage depending on the age and weight of the patient, in addition to the seriousness of the clinical condition to be treated. The exact dose and the administration route will finally be at the discretion of the physician or veterinarian.

### Experimental Section

Some examples of pharmaceutical formulations according to the invention are reported below.

### Example 1

Tablets for oral use.

Each tablet contains:
co-micronised powder containing:

| | |
|---|---|
| N-palmitoylethanolamine | 300 mg |
| Resveratrol | 25 mg |
| Resveratrol glucoside | 10 mg |
| lactose | 200 mg |
| corn starch | 100 mg |
| talc | 10 mg |
| magnesium stearate | 5 mg |
| hydroxypropylmethylcellulose | 5 mg |
| titanium dioxide | 1.5 mg |
| yellow iron oxide (E172) | 0.4 mg |

### Example 2

Capsules for oral use.

Each soft gelatine capsule contains:

| | |
|---|---|
| N-(2-hydroxyethyl)-lauroylamide | 150 mg |
| Resveratrol glucoside | 50 mg |
| soya lecithin | 30 mg |
| vegetable oil | 300 mg |
| erythrosine (E127) | 0.1 mg |

### Example 3

Lyophilised ampoules.

Each lyophilised ampoule contains:

| | |
|---|---|
| N,N′-bis-(2-hydroxyethyl)-nonandiamide | 100 mg |
| Resveratrol glucoside | 50 mg |
| mannitol | 100 mg |

Each solvent ampoule contains:

| | |
|---|---|
| pyrogen-free double distilled water | 3 ml |

### Example 4

Ampoules for intramuscular use.

Each ampoule contains:

| | |
|---|---|
| N,N′-bis-(2-hydroxyethyl)-nonandiamide | 50 mg |
| Resveratrol glucoside | 50 mg |
| physiological solution (isotonic saline) as | 2 ml |
| required up to | |

### Example 5

Cream for dermatological use.

100 g of cream contains:

| | |
|---|---|
| N,N′-bis-(2-hydroxyethyl)-nonandiamide | 2 g |
| Resveratrol | 0.5 g |
| Resveratrol glucoside | 0.3 g |
| sorbitan monostearate | 0.5 g |
| polyoxyethylene-sorbitan-monostearate | 4.5 g |
| ethyl alcohol | 3.0 g |
| stearic acid | 3.0 g |
| paraffin oil | 10.0 g |
| 70% sorbitol | 6.0 g |
| methyl p-oxybenzoate | 0.2 g |
| propyl p-oxybenzoate | 0.05 g |
| deionised water as required up to | 100.0 g |

### Example 6

Gel for gynaecological use.

100 g of gel contains:

| | |
|---|---|
| N,N′-bis-(2-hydroxyethyl)-nonandiamide | 1.0 g |
| Resveratrol glucoside | 1.0 g |
| glycerine | 8.0 g |
| Hydrogenated ricin oil | 1.0 g |
| Propylene glycol | 1.0 g |
| polycarbophil | 1.5 g |
| vitamin E acetate | 0.5 g |
| Vitamin A acetate | 0.05 g |
| polyvinyl alcohol | 0.2 g |
| hyaluronic acid | 0.2 g |
| phytosphingosine | 0.02 g |
| methyl p-oxybenzoate | 0.10 g |
| 2-phenylethanol | 0.15 g |
| quercetin | 0.1 g |
| water as required up to | 100.0 g |

### Example 7

Soft gelatin vaginal ovules.

Each ovule contains:

| | |
|---|---|
| N,N′-bis-(2-hydroxyethyl)-trans-2-dodecenediamide | 0.5 g |
| resveratrol glucoside | 0.6 g |
| propylene glycol | 1.5 g |
| erythrosine (E127) | 0.1 mg |

### Example 8

Ophthalmic ointment.

100 g of ointment contains:

| | |
|---|---|
| N,N′-bis-(2-hydroxyethyl)-trans-2-dodecenediamide | 0.5 g |
| Resveratrol | 0.5 g |
| viscous vaseline as required up to | 100.0 g |

### Example 9

Sterile ophthalmic eyewash (in single dose packs).

100 g of eyewash contains:

| | |
|---|---|
| N,N′-bis-(2-hydroxyethyl)-nonandiamide | 0.5 g |
| resveratrol glucoside | 0.3 g |
| hyaluronic acid sodium salt | 0.01 g |
| pyrogen-free physiological solution as required up to | 100.0 g |

### Example 10

Rectal suppositories.

Each suppository contains:

| | |
|---|---|
| N-palmitoylethanolamine | 0.3 mg |
| resveratrol | 0.2 mg |
| resveratrol glucoside | 0.05 mg |
| Fatty excipient for suppositories | 2.0 g |
| as required up to | |

### Example 11

Powder for chiropody use.

100 g of powder contains:

co-micronised powder containing:

| | |
|---|---|
| N,N′-bis-(2-hydroxyethyl)-lauroylamide | 2.3 g |
| resveratrol | 1.5 g |
| resveratrol glucoside | 0.8 g |
| attapulgite | 50.0 g |
| acrylic copolymer | 3.0 g |
| vitamin E acetate | 1.0 g |
| dimethicone | 2.0 g |
| isopropylmyristate | 2.0 g |
| menthol | 1.5 g |
| eucalyptol | 0.5 g |
| micronised attapulgite as required up to | 100.0 g |

### Example 12

Nail drop solution

100 g of solution contains:

| | |
|---|---|
| N,N′-bis-(2-hydroxyethyl)-nonandiamide | 2.0 g |
| N,N′-bis-(2-hydroxyethyl)-lauroylamide | 1.0 g |
| resveratrol glucoside | 2.0 g |
| phytosphingosine | 1.0 g |
| isopropyl alcohol | 80.0 g |
| acetone | 2.0 g |
| undecylenic acid | 0.2 g |
| vitamin A palmitate | 0.5 g |
| urea | 3.0 g |
| deionised water as required up to | 100.0 g |

1-19. (canceled)

20. A pharmaceutical composition comprising a mixture of:
N-palmitoylethanolamine (N-PEA), N-(2-hydroxyethyl)-lauroylamide, N,N-bis(2-hydroxyethyl)-lauroylamide, N,N′-bis(2-hydroxyethyl)nonandiamide, N,N′-bis(2-hydroyethyl)-2-dodecendiamide, mixture thereof, or a salt thereof in an amount effective to provide a dose of 0.0001 mg/Kg/day to 20 mg/Kg/day; and
resveratrol, glycoside of resveratrol, or mixture thereof in an amount effective to provide a dose of 0.005 mg/Kg/day to 10 mg/Kg/day.

21. The composition of claim 20, comprising trans isomer or cis isomer of resveratrol, glycoside of resveratrol, or mixture thereof.

22. The composition of claim 20, further comprising a pharmaceutically acceptable excipient.

23. The composition of claim 22 for oral, buccal, parenteral, rectal, transdermal, topical application onto skin and mucosa, for inhalation or insufflation.

24. The composition of claim 20, wherein the N-palmitoylethanolamine
(N-PEA), N-(2-hydroxyethyl)-lauroylamide, N,N-bis(2-hydroxyethyl)-lauroylamide, N,N′-bis(2-hydroxyethyl)nonandiamide, N,N-bis(2-hydroyethyl)-2-dodecendiamide, mixture thereof, or a salt thereof is in an amount effective to provide a dose of 0.05 mg/Kg/day to 10 mg/Kg/day.

25. A method of treating a subject suffering from a disease caused, sustained and/or characterised by the abnormal general response of the immune system in humans and animals; wherein said disease is caused by exogenous antigenic stimuli, diseases caused by endogenous antigenic stimuli (autoimmune diseases), or disease characterised and/or sustained by neuro-immunogenic inflammation, the method comprising:
administering to the subject a pharmaceutical composition comprising a mixture of:
N-palmitoylethanolamine (N-PEA), N-(2-hydroxyethyl)-lauroylamide, N,N-bis(2-hydroxyethyl)-lauroylamide, N,N′-bis(2-hydroxyethyl)nonandiamide, N,N′-bis(2-hydroyethyl)-2-dodecendiamide, mixture thereof, or a salt thereof in an amount effective to provide a dose of 0.0001 mg/Kg/day to 20 mg/Kg/day; and
resveratrol, glycoside of resveratrol, or mixture thereof in an amount effective to provide a dose of 0.005 mg/Kg/day to 10 mg/Kg/day.

26. The method according to claim 25, wherein said disease caused by exogenous antigenic stimuli is selected from the group consisting of allergic rhinitis, urticaria, atopic dermatitis, contact dermatitis, conjunctivitis, and anaphylaxis;
wherein said disease caused by endogenous antigenic stimuli (autoimmune diseases) is selected from the group consisting of: multiple sclerosis, psoriasis, bullous pemphigus, urticaria pigmentosa, systemic scleroderma, uveitis, cicatricial ocular pemphigus, rheumatoid arthritis, systemic lupus erythematosus, and psoriatic arthritis;
wherein said disease characterised and/or sustained by neuro-immunogenic inflammation is selected from the group consisting of: interstitial cystitis in humans and in cats, prostatitis, arthrosis in humans and in dogs, numerous autonomic and somatic neuropathies, vulvovaginitis, viral infections of the vagina and uterine neck, vulvar vestibolitis, oral mucositis, Crohn's disease, ulcerative colitis, geriatric dermatitis, and solar radiation or radiotherapy induced dermatitis.
